(19) United States
(12) Patent Application Publication
(10) Pub. No.: US 2011/0092896 A1
(43) Pub. Date: Apr. 21, 2011

(54) Surgical system

(75) Inventors: Christoph Kuebler, Oberkochen (DE); Martin Kraus, Huettlingen (DE); Michael Eichler, Aalen (DE); Tobias Maier, Stuttgart (DE)

(73) Assignee: Carl Zeiss Surgical GmbH

(21) Appl. No.: 12/926,609

(22) Filed: Nov. 30, 2010

Related U.S. Application Data
(63) Continuation of application No. PCT/EP2009/003715, filed on May 26, 2009.

(30) Foreign Application Priority Data
May 30, 2008 (DE) 10 2008 026 014.2

(51) Int. Cl. A61M 1/00; A61F 9/007

(52) U.S. Cl. 604/30

(57) ABSTRACT

A surgical system (1) for controlling a fluid includes an irrigation line (3), which is connected at one end to a first fluid vessel (2) for holding irrigation fluid (21) and is connected at another end to a surgical handpiece (4). The irrigation fluid (21) can be delivered at a first pressure (p1) to the handpiece (4). The surgical system further includes an aspiration inlet line (7), a second fluid vessel (15) for holding irrigation fluid (22), an aspiration venting line (14), which connects the second fluid vessel (15) to the aspiration inlet line (7), a venting valve (17), which is provided in the aspiration venting line (14) and which can switch in dependence on the fluid pressure in the aspiration inlet line (7) and/or irrigation line (3). The second fluid vessel (15) is connected to a pneumatic pressure system (23) with which the fluid (22) in the second fluid vessel (15) can be subjected to a second pressure (p2) that is higher than the first pressure (p1) in the first fluid vessel (2).

### CROSS REFERENCE TO RELATED APPLICATIONS

This application is a continuation application of international patent application PCT/EP 2009/003715, filed May 26, 2009, designating the United States and claiming priority from German application 10 2008 026 014.2, filed May 30, 2008, and the entire content of both applications is incorporated herein by reference.

### FIELD OF THE INVENTION

The invention relates to a surgical system and a method for controlling fluid during the treatment of cataract by phacoemulsification.

### BACKGROUND OF THE INVENTION

There are several surgical techniques for treatment of clouding of the lens, which is referred to in medicine as gray cataract. The most widely used technique is phacoemulsification, in which a thin tip is introduced into the diseased lens and is excited to vibration with ultrasound. In its immediate environment, the vibrating tip emulsifies the lens in such a way that the resulting lens fragments can be sucked through a line by a pump. When the lens has been completely emulsified, a new and artificial lens can be inserted into the empty capsular bag, such that a patient treated in this way can recover good visual acuity.

In phacoemulsification, a device is used that generally has a vibratable tip in a handpiece, a flushing line (irrigation line) for conveying irrigation fluid to the lens to be treated, and a suction line (aspiration line) for transporting emulsified lens fragments into a collecting vessel. During transport into the collecting vessel, it can happen that a lens fragment blocks the inlet area of the handpiece tip. With a suction pump running continuously, a vacuum therefore builds up downstream in the aspiration line. The lens fragment can be broken into smaller segments, for example by continued ultrasound vibrations of the tip, as a result of which the blockage (occlusion) is ended abruptly. The underpressure that has built up in the aspiration line has the effect that, when such an occlusion has been broken through, a relatively large amount of fluid is sucked out of the eye in a very short time. This may result in a collapse of the anterior chamber of the eye. It is then possible that the capsular bag will be drawn toward the tip of the handpiece and be punctured by the tip. In addition to such damage of the capsular bag, it is also possible for a tip that has penetrated too deeply to cause damage to the vitreous body lying behind the capsular bag.

The prior art proposes various solutions for avoiding a collapse of the anterior chamber of the eye when an occlusion is broken through. In U.S. Pat. No. 4,832,685, the aspiration line can be connected to the irrigation line, such that a pressure compensation is achieved by the irrigation fluid. A disadvantage of this is that the fluid present in the irrigation line is excited to considerable pressure fluctuations. This leads to an additional destabilization of the pressure in the anterior chamber of the eye. A further disadvantage is that, in this kind of fluid pressure compensation, contaminated fluid can flow from the aspiration line into the irrigation line. Consequently, such a surgical system can be used only for a single patient.

Another possibility is to perform pressure compensation by means of ambient air. In this case, air at atmospheric pressure is introduced into the aspiration line. However, the air introduced into the aspiration line changes the fluidic characteristics of the suction system, such that the air subsequently has to be pumped out of the aspiration line in order to once again obtain a dynamic suction pressure characteristic in the aspiration line.

U.S. Pat. Nos. 6,740,074 and 6,261,283 propose withdrawing fluid from a collecting vessel arranged at the end of the aspiration line and introducing this fluid into the aspiration line. However, contaminated particles from the collecting vessel are introduced into the aspiration line in this solution, such that a system of this kind becomes unsterile and is suitable only for a single patient, not for several patients.

### SUMMARY OF THE INVENTION

It is therefore an object of the invention to provide a surgical system which permits rapid pressure compensation when there is an underpressure in an aspiration line, wherein a sharp drop in pressure in the irrigation line is avoided at atmospheric pressure. It is also an object of the invention to provide a method for operating a surgical system of this kind.

The surgical system according to the invention for controlling a fluid includes: an irrigation line, which is connected at one end to a first fluid vessel for holding irrigation fluid, and which is connected at another end to a surgical handpiece, wherein the irrigation fluid can be conveyed at a first pressure to the handpiece, a suction pump, an aspiration inlet line, which is provided from the surgical handpiece to an inlet of the suction pump such that fluid can be suctioned through the handpiece by the suction pump, an aspiration outlet line, which connects an outlet of the suction pump to a collecting vessel in such a way that fluid can be conveyed from the outlet of the suction pump into the collecting vessel, a second fluid vessel for holding irrigation fluid, an aspiration venting line, which connects the second fluid vessel to the aspiration inlet line, a venting valve, which is provided in the aspiration venting line and can be switched as a function of the fluid pressure in the aspiration inlet line and/or irrigation line, wherein the second fluid vessel is connected to a pneumatic pressure system by means of which the fluid in the second fluid vessel can be subjected to a second pressure, which is higher than the first pressure in the first fluid vessel.

In the event of an occlusion in the aspiration line, the system according to the invention allows fluid from the second fluid vessel to be routed through the aspiration venting line into the aspiration inlet line. By means of the second pressure in the second fluid vessel, the fluid is conveyed at high speed and with a substantial pulse to the needle tip, counter to the normal fluid transport direction, and is able to force the particle blocking the needle tip out of the needle tip. The second pressure must be selected so high as to allow the particle to be forced out of the needle tip; if appropriate, the second pressure provided by the pneumatic pressure system must be increased. The particular advantage of using the pneumatic pressure system is that very rapid venting in the millisecond range is possible, while at the same time, the pressure in the irrigation line and thus also the intraocular pressure fluctuates only very slightly, if at all.

The fluid delivered does not originate from the first fluid vessel, which contains the irrigation fluid and is connected to the irrigation line. A complete separation from this first fluid vessel is achieved by the second fluid vessel, such that no direct pressure fluctuations can occur in the irrigation line during the venting procedure. Moreover, the separation of the two fluid vessels rules out the possibility of contamination of the irrigation line. Since the second fluid vessel contains sterile fluid, contamination of the aspiration line by the venting procedure is also ruled out. It is thus possible to use the surgical system on several patients treated in succession, without danger of contamination with impurities that have previously been introduced.

According to a preferred embodiment, the second fluid vessel can be filled through a filling line that has a filling valve, wherein the filling line is connected at one end to the irrigation line. It thus suffices to fill only the first fluid vessel with irrigation fluid, such that the second fluid vessel can subsequently be filled from this sterile fluid. Such filling of the second fluid vessel can take place, for example, before the start of a surgical procedure. The filling valve provides a reliable separation between the irrigation line and that part of the filling line directed toward the second fluid vessel. The filling valve also reliably separates the different fluid pressures that exist in the irrigation line and in the second fluid vessel.

The venting valve can preferably be actuated by an operator, for example manually, by means of a pedal switch or by means of an acoustic switch. The manual actuation can be effected by means of a graphic control unit or by means of a mechanical switch, for example on the handpiece. It is expedient if the operator actuates the venting valve only when an occlusion has actually taken place. However, in another embodiment, the venting valve can also be switched in such a way that it opens or closes only after a predetermined time interval has elapsed. Such an operating mode is recommended if, even in the absence of an occlusion, venting is to be carried out at regular time intervals in order to keep the aspiration line free of particles that partially or completely block the aspiration line. However, the actuation of the venting valve can also take place fully automatically, under the effect of the fluid pressures in the aspiration line or irrigation line, in which case the system according to the invention, not the operator, triggers the venting procedure.

The second pressure is preferably composed of the first pressure and of a predetermined overpressure, wherein the overpressure is a maximum of 100 mmHg. The overpressure in the eye is thus limited to a safe level.

According to another embodiment of the invention, a time period can be set in which the venting valve is switched such that fluid can pass through. The time period is preferably less than one second. This ensures that the volume of fluid conveyed to the eye through the aspiration inlet line is limited. The venting valve can preferably be switched such that a maximum fluid volume of 0.5 ml can be conveyed into the aspiration inlet line.

If the irrigation line has an irrigation valve, the latter can be brought to such a position that the irrigation line is interrupted. If the irrigation valve in the irrigation line is arranged between handpiece and filling line, the second fluid vessel can be filled particularly quickly with fluid from the first fluid vessel by way of the filling line. In this case, during the filling of the second fluid vessel, no pressure fluctuations occur in that part of the irrigation line arranged between irrigation valve and handpiece. This therefore ensures that no pressure fluctuations are induced in the eye during the filling of the second fluid vessel.

The object is also achieved by a method for controlling fluid when venting an aspiration inlet line in a surgical system as described above, in which method, after an occlusion in the aspiration inlet line, the venting valve is switched in such a way that fluid from the second fluid vessel is conveyed from the aspiration venting line to the aspiration inlet line at a second pressure. The venting valve is preferably switched at periodic intervals.

### BRIEF DESCRIPTION OF THE DRAWINGS

The invention will now be described with reference to the drawings wherein:

FIG. 1 shows a schematic of a first embodiment of the surgical system according to the invention;

FIG. 2 shows the pressure profiles in the aspiration line and irrigation line as a function of time; and,

FIGS. 3A to 3C show schematic representations of the particle movement before, during and after an occlusion when the system according to the invention is in use.

### DESCRIPTION OF THE PREFERRED EMBODIMENTS OF THE INVENTION

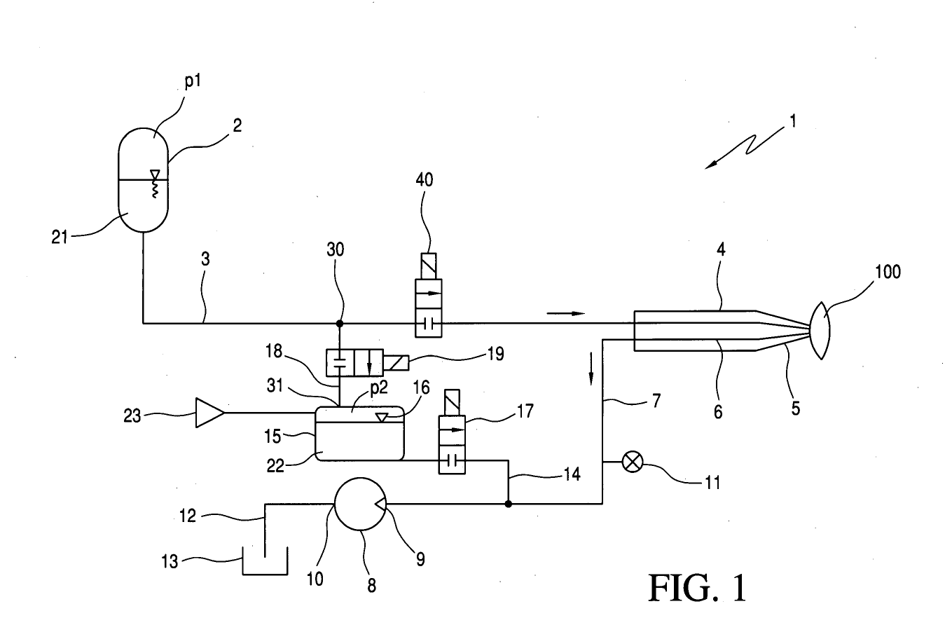

FIG. 1 is a schematic representation of an embodiment of the surgical system 1 according to the invention. A first fluid vessel 2 contains an irrigation fluid 21 that can be conveyed through an irrigation line 3 to a surgical handpiece 4. The handpiece 4 can be a phaco-emulsification handpiece in which a vibrating tip 5 emulsifies a clouded lens of an eye and the broken lens fragments are aspirated off. An irrigation valve 40, which is shown as a two-way valve in FIG. 1, enables or blocks a flow of the irrigation fluid in the direction of the handpiece 4. From the tip 5, an aspiration line 6 runs to one end of the handpiece 4 in order to transport emulsified lens fragments and fluid away from the eye. They are transported away by a suction pump 8 which, at its inlet 9, is connected to the handpiece 4 via an aspiration inlet line 7. A fluid pressure in the aspiration inlet line 7 is detected by a pressure sensor 11 which is arranged between the inlet 9 of the suction pump 8 and the handpiece 4. The pressure sensor 11 is preferably provided near the handpiece 4, such that a change of pressure in the area of the tip 5 can be detected after a short distance through the handpiece 4. A change of pressure is detected even more quickly if the pressure sensor 11 detects the fluid pressure in the aspiration line 6 inside the handpiece 4. The aspiration line 6 can be understood as a front segment of the aspiration inlet line 7 and can be formed in one piece with the aspiration inlet line 7.

The suction pump 8 conveys the lens fragments and fluid at its outlet through an aspiration outlet line 12 into a collecting vessel 13.

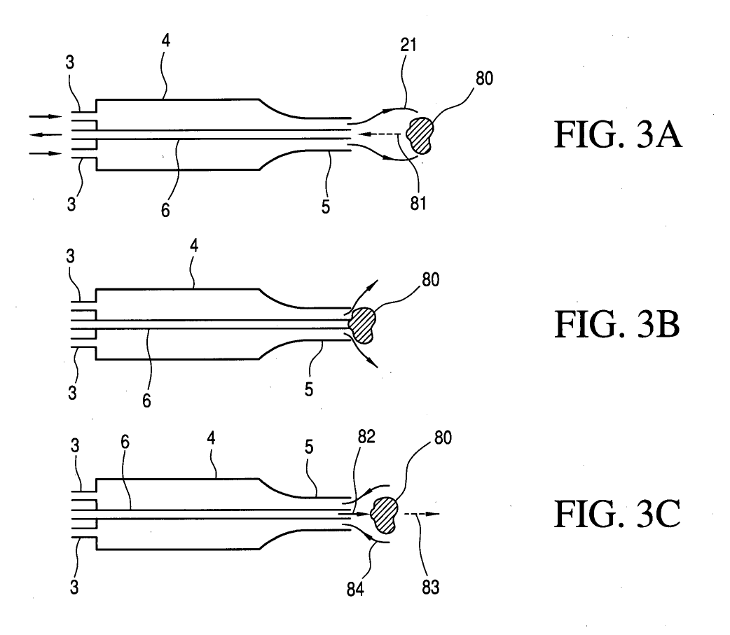

An aspiration venting line 14 connected to the aspiration inlet line 7 is connected to a second fluid vessel 15. The second fluid vessel 15 contains a fluid 22, which can be conveyed into the aspiration inlet line 7 when a two-way venting valve 17 provided in the aspiration venting line 14 is in a suitable position. If an occlusion occurs inside the aspiration line 6 or 7, for example at the distal end of the aspiration line 6 in the area of the tip 5, as a result of lens fragments that are too large, and such that suction through the aspiration lines 6 and 7 is blocked, then a vacuum pressure builds up in these lines. This pressure can be detected by the pressure sensor 11. If this vacuum pressure is present for a predetermined time and the pump is no longer suctioning any fluid, the venting valve 17 can be suitably activated. The fluid 22, which is subjected to a pressure p2 by means of a pneumatic system 23, thus flows into the aspiration venting line 14 and from there into the aspiration inlet line 7 to the needle tip 5, in order to push the particle away from the needle tip 5 by means of overpressure (see also FIGS. 3A TO 3C). When the blockage at the needle tip 5 is cleared, there is no underpressure in the eye, with the result that no dangerous suctioning of fluid from the eye takes place as in the solutions according to the prior art.

The second fluid vessel 15 is filled with a fluid 22 which, in the embodiment shown in FIG. 1, can be delivered through a filling line 18. A two-way filling valve 19 is provided in the filling line 18 and blocks or enables the through-flow of fluid. The filling line 18 is connected at one end 30 to the irrigation line 3, such that fluid 21 can be conveyed into the filling line 18. The other end 31 of the filling line 18 is connected to the fluid vessel 15. With the filling valve 19 closed, the pressure system 23 can build up an overpressure in the second fluid vessel 15 such that the fluid 22 can pass at a second pressure p2 into the aspiration venting line 14.

The second fluid vessel 15 can be provided with a sensor 16, with which the fluid level in the second fluid vessel 15 can be detected. The sensor 16 ensures that the second fluid vessel 15 is filled only as far as a maximum permitted fluid level.

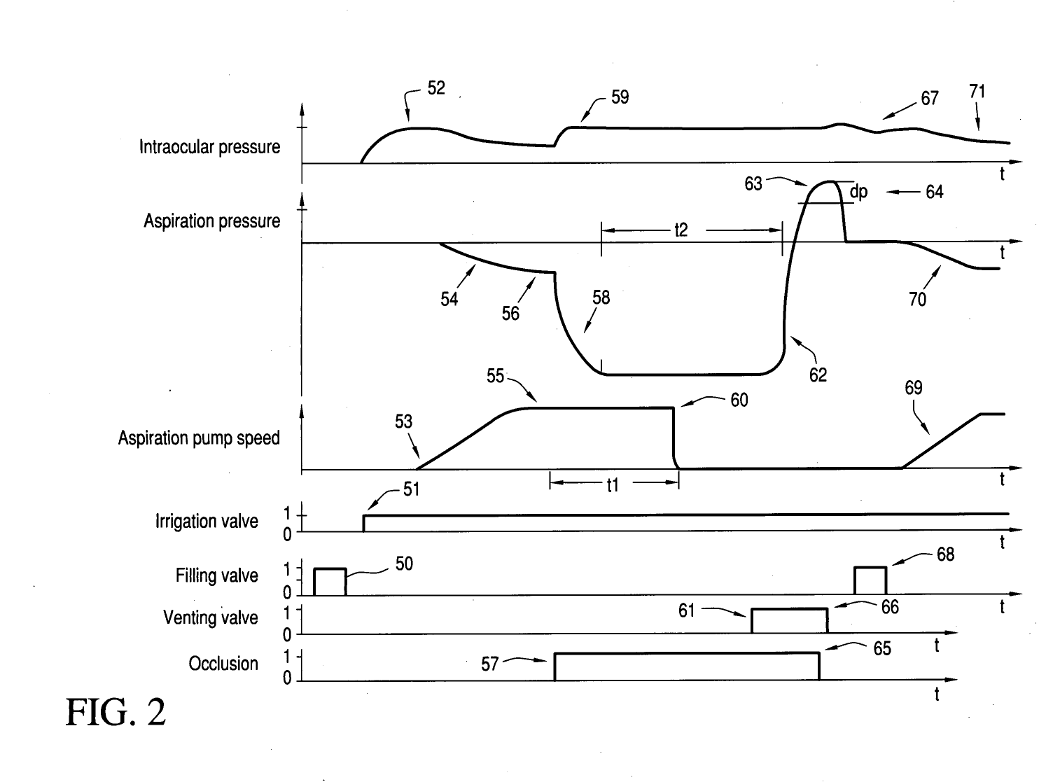

FIG. 2 is a schematic representation of the pressure profile in the aspiration line and irrigation line as a function of time (horizontal axis). Before the start of a surgical procedure, the second fluid vessel 15 is filled with irrigation fluid 22 by means of the filling valve 19 (see reference numeral 50 in FIG. 2). After the irrigation valve 40 has been opened (see reference numeral 51), irrigation fluid 21 flows through the irrigation line 3 to the handpiece 4 and from there to the lens 100 that is to be treated. The irrigation fluid 21 initially flows at a hydrostatic pressure (see reference numeral 52), which drops slightly when the suction pump 8 has been switched on (see profile of the aspiration pump speed at reference numeral 53 and the rise in the aspiration underpressure at reference numeral 54). The aspiration pump speed reached a predefined value (see reference numeral 55), such that the pressure in the aspiration line (see reference numeral 56) adjusts to a constant value.

If an occlusion takes place at the needle tip 5 in the aspiration line 6 (see reference numeral 57), the underpressure in the aspiration line 6 and in the aspiration inlet line 7 rises to a maximum attainable value at the pumping capacity (see reference numeral 58). The intraocular pressure thus rises again to the original hydrostatic value that it had at the start of the surgical procedure (see reference numerals 52 and 59). The pump is controlled such that, after the start of the occlusion, it operates for a predetermined time t1 at the same pump speed. After this time t1 has elapsed, the pump 8 is switched off, even if there is still a high vacuum pressure in the aspiration inlet line 7 (see reference numeral 60).

If the achieved vacuum pressure is present for a time t2 in which, despite the needle tip vibrating with ultrasound, the occlusion could not be broken through, it is possible, in one embodiment of the invention, to automatically switch the venting valve 17 to the through-flow position (see reference numeral 61). However, the venting valve 17 can also be actuated manually by the operator, for example after an acoustic signal caused by the drop in pressure. As a result of irrigation fluid 22 passing into the aspiration venting line 14 and aspiration inlet line 7 and also into the aspiration line 6, the vacuum pressure drops in the lines 6 and 7 (see reference numeral 62). Since the irrigation fluid 22 is conveyed at a pressure p2, which is higher than the pressure p1, the pressure in the lines 6 and 7 increases to a value that is higher than the hydrostatic pressure (see reference numeral 63). The differential pressure dp existing between p2 and p1 (see reference numeral 64) has the effect that the particle causing the occlusion in the aspiration inlet line is forced away from the needle tip, such that the occlusion is no longer present (see reference numeral 65). It will be noted that, for physiological reasons, the overpressure dp should not be more than 100 mmHg.

The venting valve 17 can be returned to the closed state (see reference numeral 66) as a function of the pressure profile in the lines 6 and 7 or only after a predetermined period of time has elapsed after the venting procedure. During the venting procedure, the intraocular pressure does not fluctuate at all or fluctuates only slightly (see reference numeral 67). The pressure fluctuation is a maximum of 20 mmHg and is considerably lower than in systems according to the prior art in which the intraocular pressure can drop to −200 mmHg. With the solution according to the invention, a dangerous underpressure of this kind in the eye no longer occurs. The quantity of fluid that is discharged from the second vessel 15 during the venting of the lines 6 and 7 can be reintroduced into the second fluid vessel 15 by brief actuation of the filling valve 19 (see reference numeral 68).

After the venting procedure, the surgical procedure can be continued again in the normal way. For this purpose, the system can start up the suction pump again until a nominal pump speed is reached (see reference numeral 69). The aspiration pressure thus likewise assumes its normal value (see reference numeral 70) and, with continued delivery of irrigation fluid, the intraocular pressure returns to the lower value that was present before the occlusion (see reference numeral 71).

FIGS. 3A to 3C show how a particle causes an occlusion and how this occlusion can be eliminated by the system according to the invention. A particle 80 drifts onto the needle tip 5, from which an irrigation fluid 21 flows (see FIG. 3A). By means of the suction pressure in the aspiration line 6, the particle 80 is drawn in the direction of the aspiration line 6 (see reference numeral 81). The particle then blocks the needle tip and the aspiration line 6 (see FIG. 3B). The irrigation fluid, which continues to flow at a pressure p1 from the needle tip, cannot remove the particle 80 from the tip. By applying the irrigation fluid flowing at a pressure p2 from the aspiration inlet line (see reference numeral 82), the particle 80 is pressed away from the needle tip 5 (see reference numeral 83 in FIG. 3C). Because of the pressure difference between irrigation line and aspiration line, fluid can enter the irrigation line (see reference numeral 84). This can lead to a slight pressure fluctuation in the irrigation line, with the intraocular pressure fluctuating only very slightly on account of the large volume of fluid in the eye. After removal of the particle at the latest, the venting valve is closed again so that the surgical procedure can be continued.

It is understood that the foregoing description is that of the preferred embodiments of the invention and that various changes and modifications may be made thereto without departing from the spirit and scope of the invention as defined in the appended claims.

What is claimed is:

1. A surgical system for controlling a fluid, the surgical system comprising:
a first fluid vessel for holding irrigation fluid;
a surgical handpiece;
an irrigation line having a first end connected to said first fluid vessel and a second end connected to said surgical handpiece and said irrigation line conducting said irrigation fluid to said surgical handpiece at a first pressure (p1);
a suction pump having an inlet and an outlet;
an aspiration inlet line extending from said surgical handpiece to said inlet of said suction pump so as to permit fluid to be drawn by suction through said surgical handpiece by said suction pump;
a collecting vessel;
an aspiration outlet line connecting said outlet of said suction pump to said collecting vessel so as to permit fluid to be conducted from said outlet into said collecting vessel;
a second fluid vessel for holding irrigation fluid;
an aspiration venting line connecting said second fluid vessel to said aspiration inlet line;
a venting valve connected into said aspiration venting line and being switchable in dependence upon the fluid pressure in said aspiration inlet line and/or in said irrigation line; and,
a pneumatic pressure system connected to said second fluid vessel for imparting a second pressure (p2) to the irrigation fluid therein which is greater than said first pressure (p1).

2. The surgical system of claim 1, further comprising a fill line for filling said second fluid vessel and said fill line having an end connected to said irrigation line; and, a fill valve connected into said fill line.

3. The surgical system of claim 1, wherein said venting valve is actuable by an operator.

4. The surgical system of claim 3, wherein said venting valve is manually actuable via a foot switch or via an acoustic switch.

5. The surgical system of claim 1, wherein the venting valve can be switched only after a predetermined period of time has elapsed.

6. The surgical system of claim 1, wherein the second pressure (p2) is composed of the first pressure (p1) and of a predetermined overpressure (dp); and, wherein said overpressure (dp) is a maximum of 100 mmHg.

7. The surgical system of claim 1, wherein a period of time can be set during which said venting valve is switched so as to cause irrigation fluid to pass therethrough.

8. The surgical system of claim 7, wherein said period of time is less than 1 second.

9. The surgical system of claim 7, wherein said venting valve can be switched so as to permit a maximum fluid volume of 0.5 ml to be conveyed into said aspiration inlet line.

10. The surgical system of claim 2, further comprising an irrigation valve connected into said irrigation line.

11. The surgical system of claim 10, wherein said irrigation valve is connected into said irrigation line between said fill line and said surgical handpiece.

12. A method for controlling fluid when venting an aspiration inlet line in a surgical system, the surgical system including: a first fluid vessel for holding irrigation fluid; a surgical handpiece; an irrigation line having a first end connected to said first fluid vessel and a second end connected to said surgical handpiece and said irrigation line conducting said irrigation fluid to said surgical handpiece at a first pressure (p1); a suction pump having an inlet and an outlet; an aspiration inlet line extending from said surgical handpiece to said inlet of said suction pump so as to permit fluid to be drawn by suction through said surgical handpiece by said suction pump; a collecting vessel; an aspiration outlet line connecting said outlet of said suction pump to said collecting vessel so as to permit fluid to be conducted from said outlet into said collecting vessel; a second fluid vessel for holding irrigation fluid; an aspiration venting line connecting said second fluid vessel to said aspiration inlet line; a venting valve connected into said aspiration venting line and being switchable in dependence upon the fluid pressure in said aspiration inlet line and/or in said irrigation line; and, a pneumatic pressure system connected to said second fluid vessel for imparting a second pressure (p2) to the irrigation fluid therein which is greater than said first pressure (p1); and, wherein an occlusion can occur in said aspiration inlet line during operation of said surgical system and said method comprising the steps of:
after the occurrence of said occlusion in said aspiration inlet line, switching said venting valve in such a manner that irrigation fluid at said second pressure (p2) from said second fluid vessel is conducted from said aspiration venting line to said aspiration inlet line.

13. The method of claim 12, wherein said venting valve is switched at periodic intervals.
